(19) United States
(12) Patent Application Publication
(10) Pub. No.: US 2023/0162859 A1
(43) Pub. Date: May 25, 2023

(54) IMPROVED METHOD FOR DETERMINING BLOOD GLUCOSE RESPONSES

(71) Applicant: Perfood GmbH, Lübeck (DE)

(72) Inventors: Christoph Twesten, Lübeck (DE); Torsten Schröder, Lübeck (DE); Fynn Marlin Leitow, Lübeck (DE)

(21) Appl. No.: 17/919,347

(22) PCT Filed: Apr. 16, 2021
(86) PCT No.: PCT/EP2021/059992

§ 371 Date: Oct. 17, 2022

(30) Foreign Application Priority Data
Apr. 17, 2020 (EP) 20170160.4

(51) Int. Cl. G16H 50/20; G16H 20/60; G16H 50/70; G16H 10/60

(52) U.S. Cl. CPC G16H 50/20; G16H 20/60; G16H 50/70; G16H 10/60

(57) ABSTRACT

The present invention pertains to a method for determining a baseline in a blood glucose curve, a method for determining a blood glucose response of an individual to at least one impact factor, a method for predicting the nutritype of an individual, a method for predicting the blood glucose response of an individual to at least one impact factor, a method for determining personalized lifestyle recommendations for an individual as well as a method for determining the composition of a personalized diet and a method for preparation thereof.

The present invention pertains to a method for determining a baseline in a blood glucose curve, a method for determining a blood glucose response of an individual to at least one impact factor, a method for predicting the nutritype of an individual, a method for predicting the blood glucose response of an individual to at least one impact factor, a method for determining personalized lifestyle recommendations for an individual as well as a method for determining the composition of a personalized diet and a method for preparation thereof.

The postprandial glycaemic response (PPGR) is a critical factor for metabolic health of humans and for the prevention and treatment of blood glucose related diseases, such as diabetes mellitus type 2 (DMT2). Numerous studies have been conducted to understand the influence of different food products on the PPGR and the health of humans. A high-glycaemic nutrition results in fast increases in blood glucose levels and in response thereto extensive secretion of insulin into the blood stream in order to absorb the glucose into the cells and convert it into glycogen. A problem arising from such excessive peaks in glucose and insulin levels is that absorption of glucose from the blood stream in response to the insulin secretin and associated therewith the reduced blood glucose levels trigger a sensation of hunger although in principle a sufficient amount of energy has already been consumed. The sensation of hunger usually results in repeated food intake, which often leads to an energy intake that exceeds the daily energy requirements of an individual and to a gradual gain of weight. Apart from this, the frequent and excessive secretion of insulin over an extended period of time in response to a high-glycaemic nutrition can lead to a downregulation of insulin receptors on the cell surface and insulin resistance. As a consequence of insulin resistance chronically increased blood glucose levels are observed resulting in various blood glucose related diseases and disorders. Nowadays, a low-glycaemic nutrition and the avoidance of excessive peaks in blood glucose levels is considered to reduce the risk for developing certain chronic diseases and to be beneficial in the treatment of a large number of diseases and disorders such as DMT2, polycystic ovary syndrome (PCOS), migraine, non-alcoholic fatty liver disease (NAFLD) and many more.

Recent studies revealed that the blood glucose level in response to ingestion of a certain food product highly depends on the individual itself, wherein general statements on responses to specific food products cannot be made (Ridaura et al., Gut microbiota from twins discordant for obesity modulate metabolism in mice, 2013, Science, Vol. 341; Zeevi et al., Personalized Nutrition by a Prediction of Glycemic Responses, 2015, Cell, Vol. 163(5)). The blood glucose response to different impact factors, such as to a certain food product, can considerably vary between different individuals. Even within a single individual, the blood glucose response depends on various aspects, such as, but not limited to daytime, general health condition and medication. The determination of the blood glucose response of an individual presently requires precise and individualized measurement of the blood glucose curve in response to a specific impact factor, such as to the consumption of a particular food product or to physical activity, under consideration of possible further interfering impact factors. The analysis and surveillance of the blood glucose response can be accomplished by using continuous blood glucose sensors and an app comprising a diary for various aspects of lifestyle, such as for recording physical activity, food intake, wellness, symptoms, pain, medication, ovulation or sleep. Based on such information, it is possible to determine personalized lifestyle recommendations, in particular a recommendation for a personalized low-glycaemic diet, to maintain or improve the health of an individual or to treat and/or prevent blood glucose related diseases and/or disorders.

At present, the standard for calculating the actual blood glucose response is based on area under the curve (AUC) calculation, which is also used for the determination of the widely used glycaemic index (GI) (Jenkins et al., Metabolic effects of low-glycemic-index diet, 1987, American Journal of Clinical Nutrition, Vol. 46(6)). Comparability of the PPGR to different food products requires a standardization of the calculation method. In various studies different methods and variants for determining the AUC have been compared (Potteiger et al., A comparison of methods for analyzing glucose and insulin areas under the curve following nine months of exercise in overweight adults, 2002, Int J. Obes. Relat. Metab. Disord., Vol. 26(1); Schnell et al., Role of Continuous Glucose Monitoring in Clinical Trials: Recommendations on Reporting, 2017, Diabetes Technol. Ther., Vol. 19(7)). According to these studies, the incremental AUC (iAUC) is suggested as a gold standard for the analysis of blood glucose curves. The blood glucose baseline describes the initial blood glucose level which is used as basis for the calculation of the AUC. It is easy to imagine that a precise and reliable determination of the AUC highly depends on the method of setting the baseline. This is often a difficult and underestimated task, in particular as in the course of the day often strong fluctuations in the blood glucose level of an individual in response to impact factors such as food intake, physical and mental activity, medication, stress, progression of a disease or the sleep pattern can be observed and frequently different factors influencing the blood glucose level are temporally overlapping.

For determination of the baseline of a blood glucose curve, usually either the glucose levels in the last hours of the night, the fasting glucose in the morning, the first determined glucose value or the daily median of the glucose level is used (Zeevi et al., Personalized Nutrition by a Prediction of Glycemic Responses, 2015, Cell, Vol. 163(5)); Brouns et al., 2005, Glycaemic index methodology, Nutrition Research Reviews, Vol. 18, pages 145-171). The use of the glucose levels in the last hours of the night is associated with various disadvantages. Blood glucose levels are influenced by the amount and quality of sleep and measurement accuracy is often negatively affected in dependency on the sleeping position. The fasting glucose in the morning also varies with quality and amount of sleep.

The methods for determining and setting of baselines for blood glucose curves in the prior art are either based on a certain blood glucose level of an individual at a specific time point or on mathematical calculation, such as calculating the average or median of the blood glucose levels measured in a certain time interval. However, these methods do not specifically consider the particular exogenous and endogenous impact factors, in particular the nature, duration and extent of the particular impact factors, causative for the progression of the blood glucose curve of an individual in a given time interval, in particular in cases in which more than one impact factor influences the progression of the blood glucose curve of an individual. Accordingly, the methods for determining a blood glucose baseline as used in the prior art do not provide robust values for correctly setting a blood glucose baseline and, therefore, do not allow precise and reliable determination of the blood glucose response of an individual to ingestion of certain food products and/or to other impact factors.

The present invention overcomes the disadvantages in the prior art by the subject-matter of the independent claims, in particular by providing an improved method for determining a baseline in a blood glucose curve, specifically an improved computer implemented method for determining a baseline in a blood glucose curve.

The present invention pertains to a method for determining a baseline in a blood glucose curve, the method comprising:
a) providing training data comprising blood glucose curves of individuals comprising an impact factor-accounting baseline,
b) subjecting the training data provided in step a) to a machine learning procedure to obtain a trained algorithm for automated determination of baselines in blood glucose curves, and
c) determining a baseline in a blood glucose curve by applying the trained algorithm on a blood glucose curve.

Thus, the present invention in particular pertains to a method of precisely and reliably determining a baseline in a blood glucose curve by using an algorithm that has been trained with training data comprising an impact factor-accounting baseline. Accordingly, the training data provided in step a) comprise blood glucose curves of individuals, wherein each of the blood glucose curves of the training data comprises a baseline that has been set taking into account the at least one impact factor affecting the individual and the corresponding progression of the blood glucose curve of the individual in response to the at least one impact factor. Preferably, each of the impact factor-accounting baselines in the blood glucose curves of the training data have specifically been determined under consideration of the at least one factor causative for a deviation of the progression of the blood glucose curve from the blood glucose curve progression which would have been obtained in absence of the at least one impact factor. Accordingly, the impact factor-accounting baselines in the blood glucose curves of the training data are not simply based on the determination of the blood glucose level of an individual at a certain time or constitute the result of an averaging of the blood glucose levels monitored during a predetermined period of time but are individually set based on information on the at least one impact factor causative for the deviation of the progression of the blood glucose curve from the blood glucose curve progression which would have been obtained in absence of the at least one impact factor, in particular on the nature, duration and extent of the at least one impact factor causative for the deviation of the progression of the blood glucose curve from the blood glucose curve progression which would have been obtained in absence of the at least one impact factor. Thus, in a preferred embodiment, the impact factor-accounting baseline is a baseline that has been obtained by assigning the effect of the at least one impact factor to the progression of the blood glucose curve of the individual and by setting a corresponding baseline not being affected by the at least one impact factor. According to the present invention, the impact factor-accounting baselines of the blood glucose curves of the training data have not been solely mathematically determined, in particular have not been determined solely based on blood glucose level measured at a specific time or solely based on averaging of measured blood glucose level. Preferably, the impact factor-accounting baseline is a baseline that has not been mathematically determined, in particular has been determined based on blood glucose level measured at a specific time or based on averaging of measured blood glucose level. In a particularly preferred embodiment, the impact factor-accounting baselines of the blood glucose curves have been determined based on expert knowledge, in particular have been determined by a qualified expert, preferably a nutritionist, a nutrition scientist and/or a medical doctor. In another preferred embodiment, the impact factor-accounting baseline is a baseline determined by at least two different qualified experts, in particular selected from a nutritionist, a nutrition scientist and/or a medical doctor. In a further preferred embodiment of the present invention, the impact factor-accounting baselines of the blood glucose curves have been determined by a subject or object able to render an independent or new opinion on the accurate progression of the baseline in a blood glucose curve. Preferably, the impact factor-accounting baselines of the blood glucose curves have been determined by a human being or by a computer. Preferably, the impact factor-accounting baselines has been determined by the human being or the computer, in particular by the qualified expert, preferably the nutritionist, the nutrition scientist and/or the medical doctor, in dependence on the effect of at least one impact factor on the progression of a blood glucose curve, in particular in dependence on information on at least one impact factor causative for the deviation of the progression of the blood glucose curve from the blood glucose curve progression which would have been obtained in absence of the at least one impact factor. According to the present invention, the training data are then subjected to a machine learning procedure in the course of which an algorithm is trained, which can automatically determine a baseline in a blood glucose curve of an individual. The trained algorithm obtained in this way is based on the recognition of certain patterns in a blood glucose curve of an individual. Particularly, the trained algorithm advantageously determines an accurate baseline accounting the specific progression of the blood glucose curve of an individual in response to at least one impact factor. The ability for this accurate determination is based on the training data used in the machine learning procedure, in particular the blood glucose curves of individuals comprising a baseline which has been set under consideration of at least one impact factor affecting an individual. In a next step of the method according to the present invention, the trained algorithm is applied on a blood glucose curve of an individual which does not comprise a blood glucose baseline so as to obtain a blood glucose curve comprising an automatically determined baseline. On the basis of the blood glucose curve of an individual comprising a baseline which has been automatically determined by the trained algorithm, it is advantageously possible to precisely and reliably calculate the blood glucose response of the individual. The determination of the blood glucose response does not suffer from the disadvantages associated with the determination methods for baselines as used in the prior art, in particular by calculating the baseline based on the glucose levels in the last hours of the night, the fasting glucose in the morning, the first determined glucose value or the daily median of the glucose level. In this way, the blood glucose response of an individual can be determined with higher accuracy, in particular also in cases in which more than one impact factor influences the blood glucose level.

In a preferred embodiment of the present invention, the training data provided in step a) comprise long-term blood glucose curves of individuals comprising an impact factor-accounting baseline, in particular blood glucose curves of individuals measured for at least 1 min, preferably at least 5 min, preferably at least 10 min, preferably at least 20 min, preferably at least 30 min, preferably at least 40 min, preferably at least 50 min, preferably at least 1 hour, preferably at least 2 hours, preferably at least 3 hours, preferably at least 4 hours, preferably at least 5 hours, preferably at least 6 hours, preferably at least 7 hours, preferably at least 8 hours, preferably at least 9 hours, preferably at least 10 hours, preferably at least 11 hours, preferably at least 12 hours, preferably at least 13 hours, preferably at least 14 hours, preferably at least 15 hours, preferably at least 16 hours, preferably at least 17 hours, preferably at least 18 hours, preferably at least 19 hours, preferably at least 20 hours, preferably at least 21 hours, preferably at least 22 hours, preferably at least 23 hours, preferably at least 24 hours, comprising an impact factor-accounting baseline.

Particularly preferred, the training data provided in step a) comprise all-day blood glucose curves of individuals comprising an impact factor-accounting baseline.

Preferably, the training data provided in step a) comprise blood glucose curves of individuals in response to at least one impact factor comprising an impact factor-accounting baseline. In a particularly preferred embodiment of the present invention, the training data provided in step a) comprise long-term blood glucose curves of individuals, in particular all-day blood glucose curves of individuals, comprising an impact factor-accounting baseline and blood glucose curves of individuals in response to at least one impact factor comprising an impact factor-accounting baseline.

Preferably, the target variables of the training data are a) blood glucose curves of individuals in response to at least one impact factor having an impact factor-accounting baseline, in particular an expert-set baseline, and b) the confidence with which a baseline can be reliably set for the blood glucose curves of individuals in response to at least one impact factor.

In a preferred embodiment of the present invention, the input variables of the training data are features derived from a) measured raw glucose values, b) information on at least one impact factor provided by the individual for the respective timeframe, and c) information on at least one individual-specific impact factor, in particular selected from age of the individual, sex of the individual, weight of the individual, height of the individual, body mass index (BMI) of the individual, waist to hip ratio, body temperature, basal metabolic rate, intestinal microbiota composition, metabolome composition, genome of the individual and/or sleeping behaviour of the individual, in particular daily sleep time and get up time.

In a further preferred embodiment of the present invention, the trained algorithm for automated determination of baselines in blood glucose curves obtained in step b) is evaluated in a step b1) based on validation data. Preferably, the validation data comprise long-term blood glucose curves of individuals, in particular all-day blood glucose curves of individuals, comprising an impact factor-accounting baseline and/or blood glucose curves of individuals in response to at least one impact factor comprising an impact factor-accounting baseline.

In a preferred embodiment of the present invention, the training data provided in step a) contain at least 10, preferably at least 50, preferably at least 100, preferably at least 250, preferably at least 500, preferably at least 750, preferably at least 1000, preferably at least 2500, preferably at least 5000, preferably at least 7500, preferably at least 10000, preferably at least 25000, preferably at least 50000, blood glucose curves of individuals, in particular pairs of blood glucose curves of individuals.

In a preferred embodiment of the present invention, the validation data used in step b1) contain at least 10, preferably at least 50, preferably at least 100, preferably at least 250, preferably at least 500, preferably at least 750, preferably at least 1000, preferably at least 2500, preferably at least 5000, preferably at least 7500, preferably at least 10000, preferably at least 25000, preferably at least 50000, blood glucose curves of individuals.

In a further preferred embodiment, the machine learning procedure is a supervised machine learning procedure.

In a particularly preferred embodiment, the machine learning procedure is based on an algorithm selected from the group consisting of linear regression, logistic regression, support vector machine, decision tree, random forest, K-nearest neighbors (kNN), K-means clustering, naive Bayes, principal component analysis (PCA), supersparse linear integer model (SLIM), neural network, gradient boosted tree regression.

In a particularly preferred embodiment of the present invention, the determination of a baseline in a blood glucose in step c) is carried out by executing the trained algorithm obtained in step b) on a blood glucose curve of an individual which does not comprise a baseline.

In a particularly preferred embodiment of the present invention, the baseline determined by the trained algorithm is an all-day baseline, in particular a baseline for the blood glucose curve of an individual observed during a single day. In a further preferred embodiment, the baseline determined by the trained algorithm is not an all-day baseline.

Preferably, the baseline determined by the trained algorithm is a baseline with regard to a particular time interval, such as a time interval of 30 min, preferably 45 min, preferably 1 hour, preferably 2 hours, preferably 3 hours, preferably 4 hours, preferably 5 hours, preferably 6 hours, preferably 7 hours, preferably 8 hours, preferably 9 hours, preferably 10 hours, preferably 11 hours, preferably 12 hours, preferably 13 hours, preferably 14 hours, preferably 15 hours, preferably 16 hours, preferably 17 hours, preferably, 18 hours, preferably 19 hours, preferably 20 hours, preferably 21 hours, preferably 22 hours, preferably 23 hours, preferably 24 hours, preferably from get up time to sleep time, preferably from sleep time to get up time.

In another preferred embodiment of the present invention, the baseline determined by the trained algorithm is a baseline for a blood glucose curve in response to a particular impact factor, such as a baseline for a blood glucose curve observed 30 min, preferably 45 min, preferably 1 hour, preferably 2 hours, preferably 3 hours, preferably 4 hours, preferably 5 hours, after a particular impact factor, such as after ingestion of a particular meal or after physical exercise.

According to further preferred embodiment of the present invention, in step c), the trained algorithm determines the quality of the automatically determined baseline, in particular determines a coefficient of determination (R²).

Preferably, the trained algorithm further determines the quality of the automatically determined all-day baseline, in particular determines a coefficient of determination (R²) for the all-day baseline.

In a further preferred embodiment of the present invention, the trained algorithm determines the quality of the automatically determined baseline with regard to a particular time interval, in particular determines a coefficient of determination (R²) for the baseline with regard to a particular time interval.

In a another preferred embodiment of the present invention, the trained algorithm determines the quality of the automatically determined baseline for a blood glucose curve in response to a particular impact factor, such as a baseline for a blood glucose curve observed 30 min, preferably 45 min, preferably 1 hour, preferably 2 hours, preferably 3 hours, preferably 4 hours, after a particular impact factor, in particular determines a coefficient of determination (R²) for the baseline for a blood glucose curve in response to a particular impact factor.

Preferably, the coefficient of determination (R²) of the baseline in the blood glucose curve determined in step c), preferably the all-day baseline, preferably the baseline with regard to a particular time interval, preferably the baseline for a blood glucose curve in response to a particular impact factor, is at least 0.8, preferably at least 0.81, preferably at least 0.82, preferably at least 0.83, preferably at least 0.84, preferably at least 0.85, preferably at least 0.86, preferably at least 0.87, preferably at least 0.88, preferably at least 0.89, preferably at least 0.9, preferably at least 0.91, preferably at least 0.92, preferably 0.93, preferably at least 0.94, preferably at least 0.95, preferably at least 0.96, preferably at least 0.97, preferably at least 0.98, preferably at least 0.99.

In a further preferred embodiment, the coefficient of determination (R²) of the baseline in the blood glucose curve determined in step c), preferably the all-day baseline, preferably the baseline with regard to a particular time interval, preferably the baseline for a blood glucose curve in response to a particular impact factor, is 0.8 to 1, preferably 0.85 to 1, preferably 0.9 to 1, preferably 0.95 to 1.

In a preferred embodiment of the present invention, the blood glucose curve in step c), in particular the blood glucose curve for which a baseline is determined by applying the trained algorithm, is a blood glucose curve of an individual in response to at least one impact factor.

In a further embodiment of the present invention, the method for determining a baseline in a blood glucose curve further comprises a step d) of analysing the blood glucose curve of the individual in response to at least one impact factor having a baseline determined by the trained algorithm, and a step e) of determining the blood glucose response of the individual to the at least one impact factor. According to this particular embodiment of the present invention, the method for determining a baseline in a blood glucose curve comprises steps a), b), c), d) and e) and is a method for determining a blood glucose response of an individual to at least one impact factor.

The invention further pertains to a method for determining a blood glucose response of an individual to at least one impact factor, in particular a computer implemented method for determining a blood glucose response of an individual to at least one impact factor, the method comprising:
aa) providing at least one blood glucose curve of the individual in response to at least one impact factor,
bb) applying the trained algorithm obtained in step b) of the method for determining a baseline in a blood glucose curve according to the present invention on the at least one blood glucose curve provided in step aa) to obtain at least one blood glucose curve of the individual having an automatically determined baseline, and
cc) analysing the at least one blood glucose curve obtained in step bb) to determine the blood glucose response of an individual to the at least one impact factor.

Accordingly, the method for determining a blood glucose response of an individual to at least one impact factor according to the present invention comprises the provision of at least one blood glucose curve of the individual in response to at least one impact factor in step aa). In a subsequent step bb), the trained algorithm obtained in step b) of the method for determining a baseline in a blood glucose curve according to the present invention is applied on the at least one blood glucose curve of the individual in response to at least one impact factor provided in step aa) to obtain at least one blood glucose curve of the individual having an automatically determined baseline. Finally, in step cc) of the method for determining a blood glucose response of an individual to at least one impact factor, the at least one blood glucose curve obtained in step bb) is analysed to determine the blood glucose response of an individual to the at least one impact factor.

In a particularly preferred embodiment of the present invention, the determination of the blood glucose response in step e) or cc) is based on calculation of the area under the curve (AUC), in particular the incremental area under the curve (iAUC), in particular under consideration of the automatically determined blood glucose baseline.

In a preferred embodiment of the present invention, the determination of the blood glucose response in step e) or cc) is based on calculation of the maximum deviation of the blood glucose curve from the automatically determined blood glucose baseline in response to the at least one impact factor, in particular the maximum increase in the blood glucose level relative to the automatically determined blood glucose baseline in response to the at least one impact factor. In a particularly preferred embodiment of the present invention, the determination of the blood glucose response in step e) or cc) is based on calculation of the postprandial peak in the blood glucose concentration with respect to the automatically determined blood glucose baseline, in particular with respect to the blood glucose baseline determined by the method for determining a baseline in a blood glucose curve according to the present invention.

In a further preferred embodiment of the present invention, the determination of the blood glucose response in step e) or cc) is based on calculation of the mathematical derivation of the blood glucose curve in response to the at least one impact factor, in particular of the slope of the blood glucose curve in response to the at least one impact factor, preferably of the steepest fall or increase of the blood glucose curve in response to the at least one impact factor.

In a preferred embodiment of the present invention, the blood glucose level of the individual is measured, preferably constantly measured, in particular by using a blood glucose sensor. As blood glucose sensor any suitable device can be used. Preferably, the blood glucose sensor is a continuous glucose monitoring (CGM) sensor, such as a Dexcom G6, Freestyle Libre or a similar device.

Preferably, the blood glucose response of the individual to at least one impact factor affecting the individual is linked to data pertaining to the at least one impact factor, preferably to data provided in a diary for various aspects of lifestyle, in particular information on daytime, duration of sleep, age of the individual, sex of the individual, weight of the individual, height of the individual, body mass index (BMI) of the individual, waist to hip ratio, body temperature, basal metabolic rate, microbiota composition in the intestinal tract of the individual, metabolome composition, genome of the individual, type of physical activity, duration of physical activity, type of mental activity, duration of mental activity, type of food, composition of food, amount of food, time of food consumption, health status, type of medication and/or dosage of medication.

In a further preferred embodiment of the present invention, the blood glucose level of the individual is measured, preferably constantly measured, for a predetermined period of time, preferably by using a blood glucose sensor, in particular a continuous glucose monitoring (CGM) sensor.

Preferably, each blood glucose response of the individual to the at least one impact factor affecting the individual during the predetermined period is linked to data pertaining to the at least one impact factor, preferably to data provided in a diary for various aspects of lifestyle, in particular information on daytime, duration of sleep, age of the individual, sex of the individual, weight of the individual, height of the individual, body mass index (BMI) of the individual, waist to hip ratio, body temperature, basal metabolic rate, microbiota composition in the intestinal tract of the individual, metabolome composition, genome of the individual, type of physical activity, duration of physical activity, type of mental activity, duration of mental activity, type of food, composition of food, amount of food, time of food consumption, health status, type of medication and/or dosage of medication.

In a preferred embodiment, the blood glucose level of the individual is measured in intervals of 30 seconds, preferably 1 minute, preferably 2 minutes, preferably 3 minutes, preferably 4 minutes, preferably 5 minutes, preferably 6 minutes, preferably 7 minutes, preferably 8 minutes, preferably 9 minutes, preferably 10 minutes, preferably 15 minutes.

Preferably, the blood glucose level of the individual is measured at least every 15 minutes, preferably at least every 10 minutes, preferably at least every 9 minutes, preferably at least every 8 minutes, preferably at least every 7 minutes, preferably at least every 6 minutes, preferably every 5 minutes, preferably every 4 minutes, preferably every 3 minutes, preferably every 2 minutes, preferably every minute, preferably every 30 seconds.

In a further preferred embodiment of the present invention, the predetermined period of time is at least 1 day, preferably at least 2 days, preferably at least 3 days, preferably at least 4 days, preferably at least 5 days, preferably at least 6 days, preferably at least 7 days, preferably at least 8 days, preferably at least 9 days, preferably at least 10 days, preferably at least 11 days, preferably at least 12 days, preferably at least 13 days, preferably at least 14 days, preferably at least 3 weeks, preferably at least 4 weeks, preferably at least 1 month, preferably at least 2 months, preferably at least 3 months, preferably at least 4 months, preferably at least 6 months, preferably at least 8 months, preferably, at least 10 months, preferably at least 12 months.

In a further preferred embodiment of the present invention, the blood glucose response of the individual to the at least one impact factor determined in step e) or cc) is included into a database comprising blood glucose responses of the individual to different impact factors, preferably into a database comprising blood glucose responses of different individuals to different impact factors, in particular a database comprising blood glucose responses of different individuals classified into the same nutritype to different impact factors.

In a particular embodiment of the present invention, the method for determining a baseline in a blood glucose curve in addition to steps d) and e) further comprises a step f) of assigning the blood glucose response of the individual to the at least one impact factor to a group of blood glucose responses of different individuals to the at least one impact factor in a database using a nutritype classification model, and a step g) of outputting the nutritype of the individual based on the at least one blood glucose response of the individual to the at least one impact factor. According to this particular embodiment of the present invention, the method for determining a baseline in a blood glucose curve comprises steps a), b), c), d), e), f) and g) and is a method for predicting the nutritype of an individual.

The present invention further relates to a method for predicting the nutritype of an individual, in particular a computer implemented method for predicting the nutritype of an individual, the method comprising:
i) providing at least one blood glucose response of an individual to at least one impact factor obtained by the method for determining a blood glucose response of an individual to at least one impact factor of the present invention,
ii) assigning the blood glucose response of the individual to the at least one impact factor to a group of blood glucose responses of different individuals to the at least one impact factor in a database using a nutritype classification model, and
iii) outputting a nutritype of the individual based on the at least one blood glucose response of the individual to the at least one impact factor.

According to the present invention, the groups of blood glucose responses of different individuals to the at least one impact factor in a database correspond to different nutritypes.

In a preferred embodiment of the present invention, the blood glucose responses to the at least one impact factor, in particular the AUC, preferably the iAUC, the maximum increase of the blood glucose level relative to the automatically determined blood glucose baseline and/or the slope of the blood glucose curve in response to the at least one impact factor, within the groups of blood glucose responses of different individuals to the at least one impact factor in the database of step f) or ii) vary from each other by at most 40%, preferably at most 35%, preferably at most 30%, preferably at most 25%, preferably at most 20%, preferably at most 18%, preferably at most 16%, preferably at most 15%, preferably at most 14%, preferably at most 13%, preferably at most 12%, preferably at most 11%, preferably at most 10%, preferably at most 9%, preferably at most 8%, preferably at most 7%, preferably at most 6%, preferably at most 5%, preferably at most 4%, preferably at most 3%, preferably at most 2%, preferably at most 2%, preferably at most 1%.

In a further preferred embodiment of the present invention, the at least one blood glucose response of the individual to at least one impact factor in step f) or i) is linked to data on food intake, in particular data on the type, composition and amount of the food consumed. Preferably, the at least one blood glucose response of the individual to at least one impact factor in step f) or i) is linked to data on at least one further impact factor, in particular to data on at least one individual-specific impact factor.

In a particularly preferred embodiment of the present invention, the method for determining a baseline in a blood glucose curve in addition to steps d) and e) further comprises a step i) of providing data pertaining to at least one impact factor, a step ii) of assigning the data pertaining to at least one impact factor to at least one blood glucose response in a database comprising blood glucose responses of the individual to different impact factors, and a step iii) of outputting a predicted blood glucose response of the individual to the at least one impact factor. According to this particular embodiment of the present invention, the method for determining a baseline in a blood glucose curve comprises steps a), b), c), d), e), i), ii) and iii) and is a method for predicting the blood glucose response of an individual to at least one impact factor.

The present invention also pertains to a method for predicting the blood glucose response of an individual to at least one impact factor, in particular a computer implemented method for predicting the blood glucose response of an individual to at least one impact factor, the method comprising:
x) providing data pertaining to at least one impact factor,
y) assigning the data pertaining to at least one impact factor to at least one blood glucose response in a database comprising blood glucose responses of the individual to different impact factors, preferably comprising blood glucose responses of different individuals to different impact factors, in particular comprising blood glucose responses of different individuals classified into the same nutritype to different impact factors, obtained by the method for determining a blood glucose response of an individual to at least one impact factor according to the present invention, and
z) outputting a predicted blood glucose response of the individual to the at least one impact factor.

In step i) or step x) of the method for predicting the blood glucose response of an individual to at least one impact factor, data on the at least one impact factor in question is provided. Preferably, the data comprises information on daytime, duration of sleep, age of the individual, sex of the individual, weight of the individual, height of the individual, body mass index (BMI) of the individual, waist to hip ratio, body temperature, basal metabolic rate, microbiota composition in the intestinal tract of the individual, metabolome composition, genome of the individual, type of physical activity, duration of physical activity, type of mental activity, duration of mental activity, type of food, composition of food, amount of food, time of food consumption, health status, type of medication and/or dosage of medication. In subsequent step ii) or y), this data is assigned to at least one blood glucose response in a database comprising blood glucose responses of the individual to different impact factors, preferably comprising blood glucose responses of different individuals to different impact factors, in particular comprising blood glucose responses of different individuals classified into the same nutritype to different impact factors, in particular wherein the blood glucose responses in the database have been obtained by the method for determining a blood glucose response of an individual to at least one impact factor according to the present invention and therefore have been determined on the basis of a blood glucose curve of the individual having an automatically determined baseline. On the basis of the assignment of the data pertaining to at least one impact factor to a specific blood glucose response of the individual to the at least one impact factor, preferably to a specific blood glucose response of a different individual to the at least one impact factor, in particular to a specific blood glucose response of a different individual classified into the same nutritype to the at least one impact factor, a prediction of the blood glucose response of the individual to the at least one impact factor is made and indicated to the individual. The predicted blood glucose response advantageously enables the individual to estimate the influence, in particular the extent and duration, of a single impact factor or a combination of various impact factors on the individual's blood glucose level. In this way, an individual can e.g. estimate the influence of consumption of a specific food at a given time on the extent and duration of the blood glucose level increase.

In case the database does not comprise a blood glucose response of the individual to the at least one impact factor in question, preferably a blood glucose response of the individual to the at least one impact factor in question or a blood glucose response of a different individual to the at least one impact factor in question, in particular a blood glucose response of a different individual classified into the same nutritype to the at least one impact factor in question, an assignment of the data to a blood glucose response of the individual to at least one impact factor, preferably to a blood glucose response of a different individual to at least one impact factor, in particular a blood glucose response of a different individual classified into the same nutritype to at least one impact factor, is made, wherein the at least one impact factor is comparable to the at least one impact factor in question based on the data pertaining to the at least one impact factor provided in step i) or x). In a particularly preferred embodiment, comparable impact factors are such which the skilled person would consider to have the most in common with the at least one impact factor in question. According to this particular embodiment, it is conceivable that the data pertaining to the impact factor in question contain the information that 200 grams of potatoes have been ingested at 5 p.m. The database, however, comprises e.g. a blood glucose response of the individual, preferably a blood glucose response of a different individual, in particular a blood glucose response of a different individual classified into the same nutritype, to the ingestion of 250 grams of potatoes at 6 p.m. In case the database does not contain any blood glucose response of the individual, preferably any blood glucose response of a different individual, in particular any blood glucose response of a different individual classified into the same nutritype, to the at least one impact factor in question, of which the corresponding at least one impact factor is closer to the at least one impact factor in question, in particular has more in common with the at least one impact factor in question, then said particular blood glucose response of the individual, preferably of a different individual, in particular a different individual classified into the same nutritype, in the database is used for the prediction of the blood glucose response of the individual to the at least one impact factor in question, in particular for the prediction of the blood glucose response of the individual to ingestion of 200 gram of potatoes at 5 p.m.

In a preferred embodiment of the present invention, the blood glucose responses of different individuals to different impact factors in the database are assigned to specific nutritypes of the individuals. According to this particular embodiment, the database comprises groups of blood glucose responses of different individuals assigned to the same nutritype.

In a preferred embodiment of the present invention, the database of step ii) or y) comprises blood glucose responses of different individuals to different impact factors, in particular blood glucose responses of different individuals classified into the same nutritype to different impact factors, obtained by the method for determining a blood glucose response of an individual to at least one impact factor according to the present invention.

Accordingly, the database of step ii) or y) can comprise blood glucose responses of the individual to different impact factors and/or blood glucose responses of different individuals to different impact factors, in particular blood glucose responses of different individuals classified into the same nutritype to different impact factors.

In a further preferred embodiment of the present invention, the database of step ii) or y) consists of blood glucose responses of the individual to different impact factors and/or blood glucose responses of different individuals to different impact factors, in particular blood glucose responses of different individuals classified into the same nutritype to different impact factors.

In a particularly preferred embodiment of the present invention, the blood glucose responses of the individual to different impact factors and/or blood glucose responses of different individuals to different impact factors, in particular blood glucose responses of different individuals classified into the same nutritype to different impact factors in the database of step ii) or y) are each linked to data on the at least one impact factor, in particular to data comprising information on daytime, duration of sleep, age of the individual, sex of the individual, weight of the individual, height of the individual, body mass index (BMI) of the individual, waist to hip ratio, body temperature, basal metabolic rate, microbiota composition in the intestinal tract of the individual, metabolome composition, genome of the individual, type of physical activity, duration of physical activity, type of mental activity, duration of mental activity, type of food, composition of food, amount of food, time of food consumption, health status, type of medication and/or dosage of medication.

In a preferred embodiment of the present invention, the blood glucose responses of different individuals to different impact factors in the database of step ii) or y) are assigned to at least two nutritypes, preferably at least three nutritypes, preferably at least four nutritypes, preferably at least five nutritypes, preferably at least six nutritypes, preferably at least seven nutritypes, preferably at least eight nutritypes, preferably at least nine nutritypes, preferably at least 10 nutritypes, preferably at least 15 nutritypes, preferably at least 20 nutritypes.

In a particularly preferred embodiment of the present invention, in a step i1) or x1) preceding step ii) or y), respectively, the individual is classified into a specific nutritype based on a nutritype classification model, in particular by the method for predicting the nutritype of an individual according to the present invention.

Preferably, in step i1) or x1) the individual is classified into a specific nutritype based on a nutritype classification model, in particular by the method for predicting the nutritype of an individual according to the present invention, assigning at least one blood glucose response of the individual to at least one impact factor to a group of comparable blood glucose responses of different individuals to the at least one impact factor in the database of step ii) or y).

In a further preferred embodiment, in step i1) or x1) the individual is classified into a specific nutritype based on a nutritype classification model, in particular by the method for predicting the nutritype of an individual according to the present invention, assigning data on at least one individual-specific impact factor to individuals having at least one identical individual-specific impact factor, preferably at least two identical individual-specific impact factors, preferably at least three identical individual-specific impact factor, preferably at least three identical individual-specific impact factors, preferably at least four identical individual-specific impact factors, preferably at least five identical individual-specific impact factors.

In a particularly preferred embodiment of the present invention, in step i1) or x1) the individual is classified into a specific nutritype based on a nutritype classification model, in particular by the method for predicting the nutritype of an individual according to the present invention, assigning data on at least one individual-specific impact factor to individuals having at least one comparable individual-specific impact factor, preferably at least two comparable individual-specific impact factors, preferably at least three comparable individual-specific impact factor, preferably at least three comparable individual-specific impact factors, preferably at least four comparable individual-specific impact factors, preferably at least five comparable individual-specific impact factors.

In a further preferred embodiment of the present invention, the nutritype classification model is obtained by a machine learning procedure, preferably by a supervised machine learning procedure, preferably by an unsupervised machine learning procedure.

Preferably, the machine learning procedure is based on an algorithm selected from the group consisting of linear regression, logistic regression, support vector machine, decision tree, random forest, K-nearest neighbors (kNN), K-means clustering, naive Bayes, principal component analysis (PCA), supersparse linear integer model (SLIM), neural network, gradient boosted tree regression.

Preferably, the comparable blood glucose responses of different individuals to the at least one impact factor, in particular the comparable blood glucose responses of different individuals classified into a specific nutritype to the at least one impact factor, have at least 50% identity, preferably at least 55% identity, preferably at least 60% identity, preferably at least 65% identity, preferably at least 70% identity, preferably at least 75% identity, preferably at least 80% identity, preferably at least 85% identity, preferably at least 90% identity, preferably at least 91% identity, preferably at least 92% identity, preferably at least 93% identity, preferably at least 94% identity, preferably at least 95% identity, preferably at least 96% identity, preferably at least 97% identity, preferably at least 98% identity, preferably at least 99% identity, preferably at least 99,5% identity, with the blood glucose responses of the individual to the at least one impact factor.

In a particularly preferred embodiment of the present invention, in step ii) or y), the data pertaining to at least one impact factor provided in step i) or x) are assigned to at least one blood glucose response in a database comprising blood glucose responses of different individuals to different impact factors, in particular in a database comprising blood glucose responses of different individuals classified into the same nutritype to different impact factors, in particular obtained by the method for determining a blood glucose response of an individual to at least one impact factor according to the present invention.

In a further preferred embodiment, the database in step ii) or y) is extended, preferably successively extended, with blood glucose response of the individual to specific impact factors, in particular determined by the method for determining a blood glucose response of an individual to at least one impact factor according to the present invention.

Preferably, the database in step ii) or y) is extended, preferably successively extended, with blood glucose response of different individuals to specific impact factors, in particular with blood glucose responses of different individuals classified into the same nutritype to specific impact factors, in particular determined by the method for determining a blood glucose response of an individual to at least one impact factor according to the present invention.

In a preferred embodiment of the present invention, the at least one impact factor is selected from food intake, physical activity, mental activity, medication, sleep or a combination thereof.

In a further preferred embodiment of the present invention, the data pertaining to at least one impact factor provided in step i) or x) include information on daytime, duration of sleep, age of the individual, sex of the individual, weight of the individual, height of the individual, body mass index (BMI) of the individual, waist to hip ratio, body temperature, basal metabolic rate, microbiota composition in the intestinal tract of the individual, metabolome composition, genome of the individual, type of physical activity, duration of physical activity, type of mental activity, duration of mental activity, type of food, composition of food, amount of food, time of food consumption, health status, type of medication and/or dosage of medication.

In a preferred embodiment of the present invention, the data pertaining to food intake include information on daytime, duration of sleep, age of the individual, sex of the individual, weight of the individual, height of the individual, body mass index (BMI) of the individual, waist to hip ratio, body temperature, basal metabolic rate, microbiota composition in the intestinal tract of the individual, metabolome composition, genome of the individual, type of physical activity, duration of physical activity, type of mental activity, duration of mental activity, type of food, composition of food, amount of food, time of food consumption, health status, type of medication and/or dosage of medication.

In a further preferred embodiment of the present invention, the data pertaining to physical activity include information on daytime, duration of sleep, age of the individual, sex of the individual, weight of the individual, height of the individual, body mass index (BMI) of the individual, waist to hip ratio, body temperature, basal metabolic rate, microbiota composition in the intestinal tract of the individual, metabolome composition, genome of the individual, type of physical activity, duration of physical activity, type of mental activity, duration of mental activity, health status, type of medication and/or dosage of medication.

In a preferred embodiment of the present invention, the data pertaining to mental activity include information on daytime, duration of sleep, age of the individual, sex of the individual, weight of the individual, height of the individual, body mass index (BMI) of the individual, waist to hip ratio, body temperature, basal metabolic rate, microbiota composition in the intestinal tract of the individual, metabolome composition, genome of the individual, type of physical activity, duration of physical activity, type of mental activity, duration of mental activity, health status, type of medication and/or dosage of medication.

In a further preferred embodiment of the present invention, the data pertaining to medication include information on daytime, duration of sleep, age of the individual, sex of the individual, weight of the individual, height of the individual, body mass index (BMI) of the individual, waist to hip ratio, body temperature, basal metabolic rate, microbiota composition in the intestinal tract of the individual, metabolome composition, genome of the individual, type of physical activity, duration of physical activity, type of mental activity, duration of mental activity, health status, type of medication and/or dosage of medication.

In a further preferred embodiment of the present invention, the data pertaining to sleep include information on daytime, duration of sleep, age of the individual, sex of the individual, weight of the individual, height of the individual, body mass index (BMI) of the individual, waist to hip ratio, body temperature, basal metabolic rate, microbiota composition in the intestinal tract of the individual, metabolome composition, genome of the individual, health status, type of medication and/or dosage of medication.

In a preferred embodiment of the present invention, the database comprises at least at least 10, preferably at least 50, preferably at least 100, preferably at least 250, preferably at least 500, preferably at least 750, preferably at least 1000, preferably at least 2000, preferably at least 3000, preferably at least 4000, preferably at least 5000, preferably at least 7500, preferably at least 10000, preferably at least 25000, preferably at least 50000, preferably at least 100000, blood glucose responses of the individual to different impact factors.

In a further preferred embodiment of the present invention, the database comprises at least at least 10, preferably at least 50, preferably at least 100, preferably at least 250, preferably at least 500, preferably at least 750, preferably at least 1000, preferably at least 2000, preferably at least 3000, preferably at least 4000, preferably at least 5000, preferably at least 7500, preferably at least 10000, preferably at least 25000, preferably at least 50000, preferably at least 100000, blood glucose responses of different individuals to different impact factors, in particular blood glucose responses of different individuals classified into the same nutritype to different impact factors.

Preferably, the database comprises blood glucose responses of the individual, preferably blood glucose responses of different individuals, in particular blood glucose responses of different individuals classified into the same nutritype, to at least two different impact factors, preferably to at least three different impact factors, preferably to at least four different impact factors, preferably to at least five different impact factors, preferably to at least six different impact factors, preferably to at least seven different impact factors, preferably to at least eight different impact factors, preferably to at least nine different impact factors, preferably to at least 10 different impact factors, preferably to at least 15 different impact factors, preferably to at least 20 different impact factors, preferably to at least 25 different impact factors, preferably to at least 30 different impact factors, preferably to at least 35 different impact factors, preferably to at least 40 different impact factors, preferably to at least 45 different impact factors, preferably to at least 50 different impact factors, preferably to at least 75 different impact factors, preferably to at least 100 different impact factors, preferably to at least 150 different impact factors, preferably to at least 200 different impact factors, preferably to at least 250 different impact factors, preferably to at least 500 different impact factors, preferably to at least 1000 different impact factors, preferably to at least 2000 different impact factors.

In a further preferred embodiment of the present invention, the database comprising blood glucose responses of the individual to different impact factors, preferably the database comprising blood glucose responses of different individuals to different impact factors, in particular the database comprising blood glucose responses of different individuals classified into the same nutritype to different impact factors, is locally stored, in particular stored on a computer-readable storage medium.

In another preferred embodiment of the present invention, the database comprising blood glucose responses of the individual to different impact factors, preferably the database comprising blood glucose responses of different individuals to different impact factors, in particular the database comprising blood glucose responses of different individuals classified into the same nutritype to different impact factors, is globally stored, in particular stored on a server.

In a preferred embodiment of the present invention, the assignment of the data pertaining to at least one impact factor provided in step i) or x) to at least one blood glucose response in a database comprising blood glucose responses of the individual to different impact factors, preferably to at least one blood glucose response in a database comprising blood glucose responses of different individuals to different impact factors, in particular to at least one blood glucose response in a database comprising blood glucose responses of different individuals classified into the same nutritype to different impact factors, in step ii) or y) is performed by a blood glucose response classification model, preferably a blood glucose response classification model obtained by a machine learning procedure.

Preferably, the machine learning procedure is an unsupervised machine learning procedure. In a further embodiment of the present invention the machine learning procedure is a supervised machine learning procedure.

Preferably, the machine learning procedure is based on an algorithm selected from the group consisting of linear regression, logistic regression, support vector machine, decision tree, random forest, K-nearest neighbors (kNN), K-means clustering, naive Bayes, principal component analysis (PCA), supersparse linear integer model (SLIM), neural network, gradient boosted tree regression.

In a preferred embodiment of the present invention, the method for determining a baseline in a blood glucose curve in addition to steps d) and e) further comprises a step aa) of providing data pertaining to at least one impact factor affecting the individual, step bb) of assigning the data pertaining to at least one impact factor affecting the individual to at least one blood glucose response in a database comprising blood glucose responses of the individual to different impact factors, step cc) of calculating personalized lifestyle recommendations for the individual based on the blood glucose response of the individual to the at least one impact factor affecting the individual in the database of step bb), and a step dd) of outputting personalized lifestyle recommendations for the individual. According to this particular embodiment of the present invention, the method for determining a baseline in a blood glucose curve comprises steps a), b), c), d), e), aa), bb), cc) and dd) and is a method for determining personalized lifestyle recommendations for an individual.

In a particularly preferred embodiment, the database of step bb) comprises blood glucose responses of different individuals to different impact factors, in particular blood glucose responses of different individuals classified into the same nutritype to different impact factors, in particular determined by the method for determining a blood glucose response of an individual to at least one impact factor according to the present invention.

The invention further pertains to a method for determining personalized lifestyle recommendations for an individual, in particular a computer implemented method for determining personalized lifestyle recommendations for an individual, the method comprising the steps:
i) providing a database comprising blood glucose responses of the individual to different impact factors, preferably a database comprising blood glucose responses of different individuals to different impact factors, in particular a database comprising blood glucose responses of different individuals classified into the same nutritype to different impact factors, determined by the method for determining a blood glucose response of an individual to at least one impact factor according to the present invention,
ii) providing data pertaining to at least one impact factor affecting the individual,
iii) calculating personalized lifestyle recommendations for the individual based on the blood glucose response of the individual to the at least one impact factor affecting the individual in the database provided in step i), preferably based on the blood glucose responses of different individuals to the at least one impact factor affecting the individual in the database provided in step i), in particular based on blood glucose responses of different individuals classified into the same nutritype to the at least one impact factor affecting the individual in the database provided in step i), and
iv) outputting personalized lifestyle recommendations for the individual.

The method for determining personalized lifestyle recommendations advantageously allows to provide an individual with a behavioural recommendation in order to avoid excessive variations or peaks in the blood glucose levels of the individual, in particular to avoid the occurrence of excessive peaks in the blood glucose curve and associated with it excessive variations or peaks of insulin and other hormones or hormonal active biomolecules and small molecules, such as (neuro-) peptides, saccharides, lipids, fatty acids, neurotransmitters, metabolites, or nucleic acids. In a preferred embodiment of the present invention, the personalized lifestyle recommendations cover various aspect of life, such as, but not limited to nutritional behaviour, physical activity and sleeping behaviour. In a particular preferred embodiment of the present invention, the personalized lifestyle recommendations are selected from the group consisting of a personalized diet, a personalized training plan, a personalized medication plan, personalized sleep recommendations and/or personalized spiritual exercises, such as meditation. The personalized lifestyle recommendations, in particular the personalized lifestyle recommendations determined by the method according to the present invention, advantageously consider the fact that the blood glucose response of different individuals to specific impact factors can considerably vary. Accordingly, the personalized lifestyle recommendations obtained in step dd) or iv) provide an individual with a behavioural recommendation for avoiding excessive variations or peaks in the blood glucose levels of the individual, preferably for maintaining or improving the health of an individual and/or for treating and/or preventing blood glucose related diseases and/or disorders.

In a preferred embodiment of the present invention, the method for determining personalized lifestyle recommendations for an individual is a method for determining the composition of a personalized diet, wherein in step cc) of the method the composition of a personalized diet based on the blood glucose response of the individual to the at least one impact factor affecting the individual in the database provided in step bb) is calculated, and wherein in step dd) the composition of a personalized diet is put out. According to this particular embodiment of the present invention, the method for determining a baseline in a blood glucose curve comprises steps a), b), c), d), e), aa), bb), cc) and dd) and is a method for determining the composition of a personalized diet.

In a further preferred embodiment of the present invention, the method for determining personalized lifestyle recommendations is a method for determining the composition of a personalized diet. According to said particular embodiment, the method comprises the steps:
i) providing a database comprising blood glucose responses of the individual to different impact factors, preferably a database comprising blood glucose responses of different individuals to different impact factors, in particular a database comprising blood glucose responses of different individuals classified into the same nutritype to different impact factors, determined by the method for determining a blood glucose response of an individual to at least one impact factor according to the present invention,
ii) providing data pertaining to at least one impact factor affecting the individual,
iii) calculating the composition of a personalized diet based on the blood glucose response of the individual to the at least one impact factor affecting the individual in the database provided in step i), preferably based on the blood glucose responses of different individuals to the at least one impact factor affecting the individual in the database provided in step i), in particular based on blood glucose responses of different individuals classified into the same nutritype to the at least one impact factor affecting the individual in the database provided in step i), and
iv) outputting the composition of a personalized diet.

Most preferably, the method for determining the composition of a personalized diet provides an individual with a dietary recommendation for a low-glycaemic nutrition, in particular with a personalized diet for use in the treatment and/or prevention of blood glucose related diseases and/or disorders.

In a particularly preferred embodiment, a selection of at least two, preferably at least three, preferably at least four, preferably at least five, different compositions of a personalized diet is provided in step dd) or iv).

Preferably, the personalized diet comprises at least one meal, preferably at least two meals, preferably at least three meals. The invention further pertains to a computer program product, directly loadable into the internal memory of a digital computer, comprising software code portions which, when the program is executed by a computer cause the computer to carry out at least one of the methods according to the present invention, in particular i) the method for determining a baseline in a blood glucose curve in response to at least one impact factor according to the present invention, ii) the method for determining a blood glucose response of an individual to at least one impact factor according to the present invention, iii) the method for predicting the nutritype of an individual according to the present invention, iv) the method for predicting the blood glucose response of an individual to at least one impact factor according to the present invention, and/or v) the method for determining personalized lifestyle recommendations for an individual according to the present invention, in particular the method for determining the composition of a personalized diet according to the present invention.

The invention further relates to a computer-readable storage medium comprising software code portions, which when executed by a computer cause the computer to carry out at least one of the methods according to the present invention, in particular i) the method for determining a baseline in a blood glucose curve in response to at least one impact factor according to the present invention, ii) the method for determining a blood glucose response of an individual to at least one impact factor according to the present invention, iii) the method for predicting the nutritype of an individual according to the present invention, iv) the method for predicting the blood glucose response of an individual to at least one impact factor according to the present invention, and/or v) the method for determining personalized lifestyle recommendations for an individual according to the present invention, in particular the method for determining the composition of a personalized diet according to the present invention.

The invention further relates to a device comprising:
a display unit, displaying a user interface,
an input unit,
a memory unit, and
a processing unit,
   wherein the memory unit comprises a computer program product according to the present invention, in particular a computer program product comprising software code portions which, when the program is executed by the processing unit cause the derive to carry out at least one of the methods according to the present invention, in particular i) the method for determining a baseline in a blood glucose curve in response to at least one impact factor according to the present invention, ii) the method for determining a blood glucose response of an individual to at least one impact factor according to the present invention, iii) the method for predicting the nutritype of an individual, iv) the method for predicting the blood glucose response of an individual to at least one impact factor according to the present invention, and/or v) the method for determining personalized lifestyle recommendations for an individual according to the present invention, in particular the method for determining the composition of a personalized diet according to the present invention.

In a preferred embodiment of the present invention, the device is a mobile device, in particular a battery-powered wireless mobile device. Preferably, the mobile device, in particular the battery-powered wireless mobile device, is selected from the group consisting of tablet computers, smartphones, smart watches and fitness tracking devices.

In a further preferred embodiment of the present invention the device, preferably mobile device, in particular battery-powered wireless mobile device, is able to establish a connection, in particular wireless connection, to a server on which a database, in particular a database comprising blood glucose responses of an individual to different impact factors, preferably a database comprising blood glucose responses of different individuals to different impact factors, in particular a database comprising blood glucose responses of different individuals classified into the same nutritype to different impact factors, is stored.

Preferably, the device, preferably mobile device, in particular battery-powered wireless mobile device, is able to introduce blood glucose responses of an individual in response to at least one impact factor, in particular blood glucose responses of an individual determined by the method for determining a blood glucose response of an individual to at least one impact factor according to the present invention, into a database comprising blood glucose responses of the individual to different impact factors, preferably a database comprising blood glucose responses of different individuals to different impact factors, in particular a database comprising blood glucose responses of different individuals classified into the same nutritype to different impact factors, stored on a server.

Thus, in a particularly preferred embodiment of the present invention, the device, preferably mobile device, in particular battery-powered wireless mobile device, is able to access and edit a database comprising blood glucose responses of the individual to different impact factors, preferably a database comprising blood glucose responses of different individuals to different impact factors, in particular a database comprising blood glucose responses of different individuals classified into the same nutritype to different impact factors, globally stored on a server.

In a further preferred embodiment of the present invention, the device, preferably desktop device or mobile device, in particular battery-powered wireless mobile device, is able to access and edit a database comprising blood glucose responses of the individual to different impact factors, preferably a database comprising blood glucose responses of different individuals to different impact factors, in particular a database comprising blood glucose responses of different individuals classified into the same nutritype to different impact factors, locally stored in the memory unit of the device.

In a particularly preferred embodiment of the present invention, the method for determining a baseline in a blood glucose curve in addition to steps d) and e) further comprises step aa) of providing data pertaining to at least one impact factor affecting the individual, step bb) of assigning the data pertaining to at least one impact factor affecting the individual to at least one blood glucose response in a database comprising blood glucose responses of the individual to different impact factors, step cc) of calculating the composition of a personalized diet based on the blood glucose response of the individual to the at least one impact factor affecting the individual in the database provided in step bb), step dd) of outputting the composition of a personalized diet, and in addition thereto step ee) of preparing the components of the personalized diet having the composition calculated in step cc). According to this particular embodiment of the present invention, the method for determining a baseline in a blood glucose curve comprises steps a), b), c), d), e), aa), bb), cc), dd) and ee) and is a method for preparing a personalized diet.

The invention also pertains to a method for preparing a personalized diet, the method comprising the steps:
xx) determining the composition of a personalized diet according to the method for determining a composition of a personalized diet according to the present invention, and
yy) preparing the components of the personalized diet having the composition determined in step xx).

The invention further pertains to a personalized diet obtained by the method according to the present invention, in particular in step ee) of the method according to the present invention or by the method for preparing a personalized diet, wherein the personalized diet is for use in the treatment of blood glucose related diseases and/or disorders.

In a preferred embodiment of the present invention, the blood glucose related disease and/or disorder is selected from the group consisting of diabetes mellitus type 1 (DMT1), diabetes mellitus type 2 (DMT2), gestational diabetes, hyperglycaemia, metabolic syndrome, cardiovascular diseases, glucose intolerance, polycystic ovary syndrome (PCOS), migraine, non-alcoholic fatty liver disease (NAFLD), cancer, acne, atopic dermatitis, psoriasis, rosacea, atrial fibrillation, dyslipidaemia, HIV, arterial hypertension, pre-diabetes, obesity, brain/cognitive dysfunction, Alzheimer's disease, depression, symptoms of menopause, menstrual dysregulation, cartilage damage, Parkinson's disease, rheumatic diseases, chronic inflammation.

The invention also pertains to the use of a personalized diet obtained by the method according to the present invention, in particular in step ee) of the method according to the present invention or by the method for preparing a personalized diet according to the present invention, in induction of pregnancy, regulation of menstrual cycle, weight loss, anti-aging or in treatment and/or prevention of menstrual problems and symptoms of menopause.

In a preferred embodiment of the present invention, the database comprising blood glucose responses of the individual to different impact factors, preferably the database comprising blood glucose responses of different individuals to different impact factors, in particular the database comprising blood glucose responses of different individuals classified into the same nutritype to different impact factors, is a database comprising blood glucose responses determined based on an automatically set baseline, in particular based on a baseline which has been set using the method for determining a baseline in a blood glucose curve according to the present invention. Preferably, the database does not comprise blood glucose responses based on a baseline which has solely been mathematically determined, in particular which has solely been determined based on a blood glucose level measured at a specific time or which is solely based on averaging of measured blood glucose levels. In a further preferred embodiment, the database does not comprise blood glucose responses based on a baseline which has been mathematically determined, in particular which has been determined based on a blood glucose level measured at a specific time or which is based on averaging of measured blood glucose levels.

In a further preferred embodiment of the invention, the blood glucose response of the individual to at least one impact factor determined in steps a), b), c), d) and e) is introduced into the database comprising blood glucose responses of the individual to different impact factors, preferably the database comprising blood glucose responses of different individuals to different impact factors, in particular the database comprising blood glucose responses of different individuals classified into the same nutritype to different impact factors, after step e). According to this embodiment of the present invention, the method comprises a step e2) of introducing the blood glucose response of the individual to the at least one impact factor determined in step e) into database comprising blood glucose responses of the individual to different impact factors, preferably the database comprising blood glucose responses of different individuals to different impact factors, in particular the database comprising blood glucose responses of different individuals classified into the same nutritype to different impact factors.

Preferably, the blood glucose response of the individual to at least one impact factor determined in steps a), b), c), d) and e) is introduced into the database comprising blood glucose responses of the individual to different impact factors, preferably the database comprising blood glucose responses of different individuals to different impact factors, in particular the database comprising blood glucose responses of different individuals classified into the same nutritype to different impact factors, before step f), ii) and/or bb), preferably before step f), preferably before step ii), preferably before step bb). Accordingly, the database used in step f), ii) and/or bb), preferably step f), preferably step ii), preferably step bb), preferably comprises the blood glucose response of the individual to at least one impact factor determined in steps a), b), c), d) and e), in particular determined based on a blood glucose curve of an individual in response to at least one impact factor having a baseline determined by a trained algorithm, in particular determined by the method for determining a baseline in a blood glucose curve according to the present invention. According to this embodiment of the present invention, the method comprises a step f1), ii1) and/or bb1), preferably step f1), preferably step ii1), preferably step bb1), of introducing the blood glucose response of the individual to the at least one impact factor determined in step e) into database comprising blood glucose responses of the individual to different impact factors, preferably the database comprising blood glucose responses of different individuals to different impact factors, in particular the database comprising blood glucose responses of different individuals classified into the same nutritype to different impact factors, wherein step f1), ii1) and/or bb1), preferably step f1), preferably step ii1), preferably step bb1), is conducted before step f), ii) and/or bb), preferably before step f), preferably before step ii), preferably before step bb).

In the context of the present invention, the term “blood glucose curve” refers to the concentration of glucose in the blood in a time dependent manner. Particularly, a “blood glucose curve” is a plot of blood glucose levels measured at various time points which is usually depicted in a coordinate system (x-axis: time, y-axis: glucose level). The term “blood glucose level” describes the concentration of glucose in the blood at a given time. In the context of the present invention, the term “blood glucose response” often also referred to as “glycaemic response” designates the time dependent progression of the blood glucose curve in response to at least one specific impact factor under consideration of the blood glucose baseline of an individual. The “blood glucose response” preferably encompasses information on the exact time dependent progression and extent of the blood glucose curve assignable to at least one particular impact factor. In a particularly preferred embodiment, the “blood glucose response” corresponds to the area under curve (AUC), in particular the incremental area under the curve (iAUC), which is bordered by the blood glucose baseline, the maximum increase in the blood glucose level relative to the automatically determined blood glucose baseline or the slope of the blood glucose curve, in particular the steepest fall or increase of the blood glucose curve in response to the at least one impact factor.

In the context of the present invention, the term “impact factor” relates to any exogenous and endogenous factor suitable to influence the blood glucose level of an individual, in particular suitable to trigger a blood glucose response of an individual. The term “individual-specific impact factor” as used in the context of the present invention relates to specific endogenous factors of an individual suitable to influence the blood glucose level, such as, but not limited to age of the individual, sex of the individual, weight of the individual, height of the individual, body mass index (BMI) of the individual, waist to hip ratio, body temperature, basal metabolic rate, intestinal microbiota composition, metabolome composition, genome of the individual, sleeping behaviour of the individual, in particular daily sleep time and get up time.

The term “algorithm” designates a sequence of well-defined unambiguous computer-implementable instructions to perform a specific task.

In the context of the present invention, the expression “nutritype classification model” designates a classification model classifying an individual into a specific nutritype.

The expression “blood glucose response classification model” as used in the context of the present invention relates to a classification model assigning data pertaining to at least one impact factor to at least one blood glucose response in a database comprising blood glucose responses of the individual to different impact factors, preferably comprising blood glucose responses of different individuals to different impact factors, in particular blood glucose responses of different individuals classified into the same nutritype to different impact factors.

In the context of the present invention, the term “nutritype” designates a defined group of individuals which are characterized by a comparable blood glucose response to specific impact factors, in particular to a group of individuals which are characterized by a comparable metabolism. Preferably, the individuals of a specific “nutritype” are characterized by a similar genotype, are epigenetically similar and/or have comparable compositions of the intestinal microbiome.

In the context of the present invention, the expression “blood glucose curve having an impact factor-accounting baseline” designates a blood glucose curve comprising a baseline, which has been determined under consideration of the at least one impact factor causative for a deviation of the progression of the blood glucose curve from the blood glucose curve progression which would have been obtained in absence of the at least one impact factor, in particular in dependence on the at least one impact factor causative for a deviation of the progression of the blood glucose curve from the blood glucose curve progression which would have been obtained in absence of the at least one impact factor. Particularly, an “impact factor-accounting baseline” is in contrast to the blood glucose curve baselines of the prior art preferably not simply based on the determination of the blood glucose level of an individual at a certain time or constitutes the result of averaging of the blood glucose levels monitored during a predetermined time period but is preferably individually set based on information on the at least one impact factor causative for the deviation of the progression of the blood glucose curve from the blood glucose curve progression which would have been obtained in absence of the at least one impact factor, in particular on the nature, duration and extent of the at least one impact factor causative for the deviation of the progression of the blood glucose curve from the blood glucose curve progression which would have been obtained in absence of the at least one impact factor. Preferably, the impact factor-accounting baseline is a baseline that has been obtained by assigning the effect of the at least one impact factor to the progression of the blood glucose curve of the individual and by setting a corresponding baseline not being affected by the at least one impact factor. The “impact factor-accounting baseline” according to the present invention is a baseline, which is not solely mathematically determined, in particular which is not solely determined based on a blood glucose level measured at a specific time or solely based on averaging of measured blood glucose level. Particularly preferred, the “impact factor-accounting baseline” according to the present invention is a baseline, which is not mathematically determined, in particular which is not determined based on a blood glucose level measured at a specific time or based on averaging of measured blood glucose level. Preferably, the “impact factor-accounting baseline” is a baseline, which has been determined based on expert knowledge, in particular has been determined by a qualified expert, preferably a nutritionist, a nutrition scientist and/or a medical doctor. In a further preferred embodiment, the “impact factor-accounting baseline” is a baseline determined by at least two different qualified experts, in particular selected from a nutritionist, a nutrition scientist and/or a medical doctor. In a further preferred embodiment of the present invention, the “impact factor-accounting baselines” of the blood glucose curves have been determined by a subject or object able to render an independent or new opinion on the accurate progression of the baseline in a blood glucose curve. Preferably, the “impact factor-accounting baselines” of the blood glucose curves have been determined by a human being or by a computer. Preferably, the “impact factor-accounting baselines” has been determined by the human being or the computer, in particular by the qualified expert, preferably the nutritionist, the nutrition scientist and/or the medical doctor, in dependence on the effect of at least one impact factor on the progression of a blood glucose curve, in particular in dependence on information on at least one impact factor causative for the deviation of the progression of the blood glucose curve from the blood glucose curve progression which would have been obtained in absence of the at least one impact factor. According to the present invention, the conduction of the analysis and/or the determination, in particular of the non-mathematical analysis and/or determination, of a baseline in a blood glucose curve based on expert knowledge, in particular based on human expertise, as such is not part of the present invention. The methods according to the present invention are based on the use of training data comprising blood glucose curves of individuals comprising an “impact factor-accounting baseline” which has previously been determined, in particular not solely mathematically determined, preferably not mathematically determined, wherein these training data are used for training an algorithm for automated baseline determination by employing a machine learning procedure.

In the context of the present invention, the terms “food” and “food product” encompass any raw and prepared comestible product, such as specific fruits, vegetables, meat, fish, as well as combinations of different raw and prepared comestible products, such as bread, breakfast meals, pasta dishes, salads, sauces, beverages, confectionery, candy. The terms further relate to at least one micro- or macro-nutrient and combinations of micro- and macro-nutrients.

In the context of the present invention the term “personalized” pertains to an individual-specific adaptation, in particular an individual-specific adaptation considering the exogenous and endogenous factors affecting a particular individual. Accordingly, the term “personalized diet” as used in the context of the present invention relates to a nutrition specifically adapted to a particular individual under consideration of the exogenous and endogenous factors affecting the individual.

The term “personalized lifestyle recommendations” pertains to tailored recommendations for an individual's way or style of life, in particular for the behaviour of the individual. In the context of the present invention the term may cover various aspect of life, such as, but not limited to nutritional behaviour, physical activity and sleeping behaviour. The “personalized lifestyle recommendations” according to the present invention particularly aims to avoid excessive variations or peaks in the blood glucose levels of the individual.

In the context of the present invention, the expression “determining the composition of a personalized diet” pertains to the determination/prediction of the compounds making up a personalized diet, in particular the nutrition of an individual tailored to the individual's specific blood glucose responses to the separate nutritional compounds. The determination of the composition of a personalized diet therefore serves to allow compiling components of an individual's nutrition, such as the components of a meal, in dependency on the individual's specific blood glucose responses. A personalized diet having the composition as determined by the method according to the present invention advantageously allows to avoid excessive variations or peaks in glucose and insulin levels of an individual in response to the consumption of meals. The expression “determining the composition of a personalized diet” is therefore not directed to the chemical analysis of a given diet but to the determination/prediction of the composition of an individual's diet which does not lead to excessive variations or peaks in glucose and insulin levels of an individual.

The term “personalized diet” encompasses a personalized single meal, but also a recommendation of various personalized meals to be preferably consumed during a day, week or month. The term further relates to personalized compositions of micro- and macro-nutrients.

In the context of the present invention the term “computer-readable storage medium” includes any machine readable medium, in particular computer storage media and communication media including any medium that facilitates transfer of a computer program from one place to another. A storage media may be any available media that can be accessed and read by a computer. By way of example, and not limitation, such computer-readable media can comprise random access memory (RAM), read-only memory (ROM), electrically erasable programmable read-only memory (EEPROM), or other optical disk storage, semiconductor memory, magnetic disk storage or any other medium that can be used to carry or store desired program code in the form of instructions or data structures and that can be accessed by a computer. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, or wireless technologies such as infrared, radio, and microwave are included in the definition of medium. Disk and disc, as used herein, includes compact disc (CD), laser disc, optical disc, digital versatile disc (DVD), floppy disk and blu-ray disc, wherein disks usually reproduce data magnetically, while discs reproduce data optically with lasers.

In the context of the present invention, the term “a” is meant to include the meaning of “one” or “one or more”.

In the context of the present invention, the term “comprising” preferably has the meaning of “containing” or “including”, meaning including the specifically identified elements without excluding the presence of further elements. However, in a preferred embodiment, the term “comprising” is also understood to have the meaning of “consisting essentially of” and in a further preferred embodiment the meaning of “consisting of”.

Further preferred embodiments of the invention are subject of the following aspects and of the subclaims.

Aspect 1: A method for determining a baseline in a blood glucose curve, the method comprising:
a) providing training data comprising blood glucose curves of individuals comprising an impact factor-accounting baseline,
b) subjecting the training data provided in step a) to a machine learning procedure to obtain a trained algorithm for automated determination of baselines in blood glucose curves, and
c) determining a baseline in a blood glucose curve by applying the trained algorithm on a blood glucose curve in response.

Aspect 2: The method of aspect 1, wherein the training data provided in step a) contain at least 10, preferably at least 50, preferably at least 100, preferably at least 250, preferably at least 500, preferably at least 750, preferably at least 1000, pairs of blood glucose curves in response to at least one impact factor.

Aspect 3: The method according to any one of the preceding aspects, wherein the machine learning procedure is a supervised machine learning procedure.

Aspect 4: A method for determining a blood glucose response of an individual to at least one impact factor, the method comprising:
aa) providing at least one blood glucose curve of the individual in response to at least one impact factor,
bb) applying the trained algorithm obtained in step b) of the method according to any one of aspects 1 to 3 on the at least one blood glucose curve provided in step aa) to obtain at least one blood glucose curve of the individual having an automatically determined baseline, and
cc) analysing the at least one blood glucose curve obtained in step bb) to determine the blood glucose response of an individual to the at least one impact factor.

Aspect 5: A method for predicting the nutritype of an individual, the method comprising:
i) providing at least one blood glucose response of an individual to at least one impact factor obtained by the method according to aspect 4,
ii) assigning the blood glucose response of the individual to the at least one impact factor to a group of blood glucose responses of different individuals to the at least one impact factor in a database using a nutritype classification model, and
iii) outputting the nutritype of the individual based on the at least one blood glucose response of the individual to the at least one impact factor.

Aspect 6: A method for predicting the blood glucose response of an individual to at least one impact factor, the method comprising:
x) providing data pertaining to at least one impact factor,
y) assigning the data pertaining to at least one impact factor to at least one blood glucose response in a database comprising blood glucose responses of the individual to different impact factors obtained by the method according to aspect 4, and
z) outputting a predicted blood glucose response of the individual to the at least one impact factor.

Aspect 7: The method according to any one of the preceding aspects, wherein the at least one impact factor is selected from food intake, physical activity, mental activity, stress, health condition medication, sleep or a combination thereof.

Aspect 8: A computer program product, directly loadable into the internal memory of a digital computer, comprising software code portions which, when the program is executed by a computer cause the computer to carry out i) the method according to any one of aspects 1 to 3, ii) the method according to aspect 4, iii) the method according to aspect 5, iv) the method according to aspect 6 and/or v) the method according to aspect 12.

Aspect 9: A computer-readable storage medium comprising software code portions, which when executed by a computer cause the computer to carry out i) the method according to any one of aspects 1 to 3, ii) the method according to aspect 4, iii) the method according to aspect 5, iv) the method according to aspect 6 and/or v) the method according to aspect 12.

Aspect 10: A device comprising:
a display unit, displaying a user interface,
an input unit,
a memory unit, and
a processing unit,
   wherein the memory unit comprises a computer program product according aspect 8.

Aspect 11: The device according to aspect 10, wherein the device is a mobile device, in particular a battery-powered wireless mobile device.

Aspect 12: A method for determining personalized lifestyle recommendations for an individual, the method comprising the steps:
i) providing a database comprising blood glucose responses of the individual to different impact factors determined by the method according to aspect 4,
ii) providing data pertaining to at least one impact factor affecting the individual,
iii) calculating personalized lifestyle recommendations for the individual based on the blood glucose response of the individual to the at least one impact factor affecting the individual in the database provided in step i), and
iv) outputting personalized lifestyle recommendations for the individual.

Aspect 13: The method according to aspect 12, wherein the method is a method for determining the composition of a personalized diet, wherein in step iii) the composition of a personalized diet based on the blood glucose response of the individual to the at least one impact factor affecting the individual in the database provided in step i) is calculated, and wherein in step iv) the composition of a personalized diet is put out.

Aspect 14: A method for preparing a personalized diet, the method comprising the steps:
xx) determining the composition of a personalized diet according to the method according to aspect 13, and
yy) preparing the components of the personalized diet having the composition determined in step xx).

Aspect 15: A personalized diet obtained by the method of aspect 14 for use in the treatment of blood glucose related diseases and/or disorders.

Aspect 16: The personalized diet for use according to aspect 14, wherein the blood glucose related disease and/or disorder is selected from the group consisting of diabetes mellitus type 1 (DMT1), diabetes mellitus type 2 (DMT2), gestational diabetes, hyperglycaemia, metabolic syndrome, cardiovascular diseases, glucose intolerance, polycystic ovary syndrome (PCOS), migraine, non-alcoholic fatty liver disease (NAFLD), cancer, acne, atopic dermatitis, psoriasis, rosacea, atrial fibrillation, dyslipidaemia, HIV, arterial hypertension, pre-diabetes, obesity, brain/cognitive dysfunction, Alzheimer's disease, depression, cartilage damage, Parkinson's disease, rheumatic diseases, chronic inflammation.

Aspect 17: Use of a personalized diet obtained by the method of aspect 14 in induction of pregnancy, regulation of menstrual cycle, weight loss, anti-aging or in treatment and/or prevention of menstrual problems and symptoms of menopause. The present invention is further illustrated by way of the following examples and figures.

### EXAMPLE 1

### Training an Algorithm for Determining Blood Glucose Baselines

1. Training Data

In order to train an algorithm for the determining of blood glucose baselines, the following data is used:
target variables:
   expert-set baselines for one day (in mg/dL)
   the confidence with which a baseline can be reliably set for the whole day and used for the evaluation of all meals for that day (0-100%)
input variables are features derived from:
   measured raw glucose values
   app entries made by the participant for the same timeframe
   a digital anamnesis

The data is aggregated for the mentioned sources and stored in a SQL database for evaluation.

1.1 Baselines:

For each day with sufficient (>8 h) present glucose measurements, the measured glucose values are plotted (x-axis: time, y-axis: glucose level) and presented to nutrition experts via a web-based tool. This tool allows to:
set a horizontal line as a baseline for the currently viewed day. The glucose level on the y-axis for this horizontal line is then saved and used for the meal evaluations of this day.
set a meal specific baseline which will be used instead of the day baseline
mark the data for the whole day or for a single meal as invalid due to measurement errors.

The nutrition experts were qualified as nutritionist, nutrition scientist or medical doctor. Each day is evaluated by two experts. If the experts' agreement is within 5 mg/dl, the mean of the baselines is used. If not, a third expert evaluates this day and attempts to resolve the disagreement. If the three experts can agree on one baseline, this value is used. Otherwise, it is marked as invalid and is not used for training the algorithm.

1.2 Glucose Data:

Data from a single day (00:00-23:59) within a 14-day measurement period using a CGM sensor, such as Dexcom G6, Freestyle Libre or a similar device, serves as raw input. Based on the daily values, descriptive statistic and meal specific features are computed.

The following features are computed from the glucose values of a day:
the first three descriptors of a Fourier transformation. Features derived from other transformations (e.g. Laplace-Transform) can also be used.
the mean, median maximum and minimum of the whole day, of the times without meal/activity (+2 h) or sleep and of the 3 h before waking up.

The following is computed from the 2 h window after ingesting a meal:
the mean of estimated meal specific baselines.
These estimates for meal specific baselines are approximated from y-intercepts obtained by polynomial interpolations, Taylor series or other curve fitting methods like data assimilation via bayesian methods based on mathematical models for the postprandial glucose response.

1.3 Logged Entries:

During the measurement period, the participants are encouraged to actively log eaten meals, physical activity, taken medication and their daily sleep time and get up time. From these entries, the following features are computed:
number and daily distribution of calories ingested by meals and burned by physical activity
the total amount of calories ingested via meals and burned via sport after 18:00 on the previous day
the sleep quality and sleep duration from the night before the evaluated day
the time from get up until the first meal
the number of MET-minutes derived from the logged activities for the day
the number of minutes and without sleep, activity or a meal +2 h for the day
whether a medication that is known to affect the blood sugar level has been taken

1.4 Anamnesis:

Customers fill out a digital anamnesis in an app when starting the programme, providing information about their physiology. The following features are provided by or computed from the anamnesis:
age
sex
body mass index
waist to hip ratio
usual sleeping duration (usual bed- and get up time)
basal metabolic rate

1.5 Dataset Size:

71.432 blood glucose curves of individuals with expert-set baselines have been used for the training of the final algorithm after pre-processing. These baselines were annotated for 4.880 runs of the program, i.e. measurement periods for a participant of 14 days (occasionally longer when a blood glucose sensor needed replacement).

In total, 28.454 meals with per-meal baselines and 458.531 meals with day baselines assigned by 105.892 day baselines are used as raw data set.

2. Model Training

2.1 Algorithm Choice

Machine learning algorithms such as Random Forest or Gradient Boosted Tree Regression or others are used to predict the target variable (the day baseline) based on the input features derived from the glucose data, the logged entries and the anamnesis.

2.2 Selection

Techniques like grid search for hyperparameter tuning and iterating over the different subsets of model features are used in order to select the final model, optimising for the coefficient of determination (R² score) for the agreement of the predicted values with the annotated baselines after performing a split of the data set into testing, training and validation sets. The final R² score was 0.91.

### EXAMPLE 2

### Application of the Trained Algorithm

For each new customer, the trained algorithm determines the day baseline and computes the certainty of this determination (i.e. the probability with which this day will neither be marked as invalid nor require a meal-specific baseline).

Each baseline determined by the trained algorithm can be approved as quality control mechanism before it is used for the determination of a blood glucose response by AUC calculation (see “baselines”). In an alternative embodiment, only those baselines with a low certainty are approved before their use for the AUC calculation.

### EXAMPLE 3

### Determination of the Blood Glucose Response of an Individual

The blood glucose response of an individual to ingestion of a meal has been determined based on a blood glucose curve comprising a blood glucose baseline determined by the trained algorithm of Example 1 by calculating the difference of the maximum of the glucose curve within 2 hours after starting the meal and the blood glucose baseline.

The iAUC can be computed as the area under the curve between the blood glucose curve and the blood glucose baseline within 2 hours after starting the meal.

The slope can be computed as the steepest fall of the glucose curve within 2 hours after the start of the meal.

### EXAMPLE 4

### Nutritype Determination

The nutritype of an individual is computed from the blood glucose responses to certain foods. For example, for the whitebread/wholemeal bread nutritype the individual eats whitebread and wholemeal bread at different days but at the same time of the day. Both meals are sized such that they contain the same amount of carbohydrates. An individual with a significantly lower blood glucose response to the wholemeal bread will then be labelled as wholemeal bread type.

### FIGURES

FIG. 1 shows a flowchart of the individual steps of the method for determining a baseline in a blood glucose curve according to the present invention. Initially, an algorithm is trained to automatically determine baselines in blood glucose curves based on training data comprising blood glucose curves of individuals having an impact factor-accounting baseline. Subsequently, the trained algorithm is applied on a blood glucose curve of an individual to obtain a blood glucose curve having an automatically determined baseline.

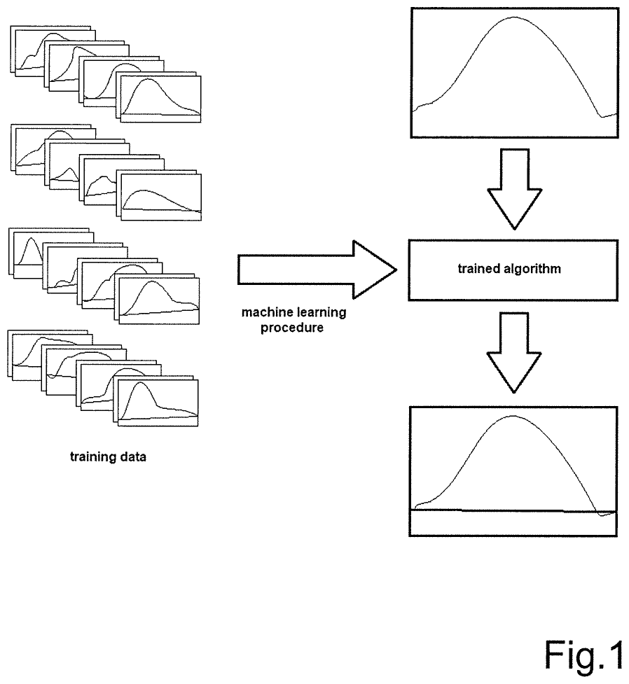

FIG. 2 shows a flowchart of the individual steps of the method for determining a blood glucose response of an individual to at least one impact factor according to the present invention. In the first step of the method, at least one blood glucose curve of an individual in response to at least one impact factor is provided. Subsequently, the trained algorithm obtained by the method for determining a baseline in a blood glucose curve in response to at least one impact factor according to the present invention is applied on the at least one blood glucose curve of the individual so as to obtain a blood glucose curve having an automatically determined baseline. Finally, the obtained blood glucose curve having an automatically determined baseline is analysed to determine the blood glucose response of the individual to the at least one impact factor, such as by calculating the AUC, in particular the iAUC (A; grey shaded), by determining the maximum increase in the blood glucose level relative to the automatically determined blood glucose baseline (B) or by determining the slope of the blood glucose curve (C).

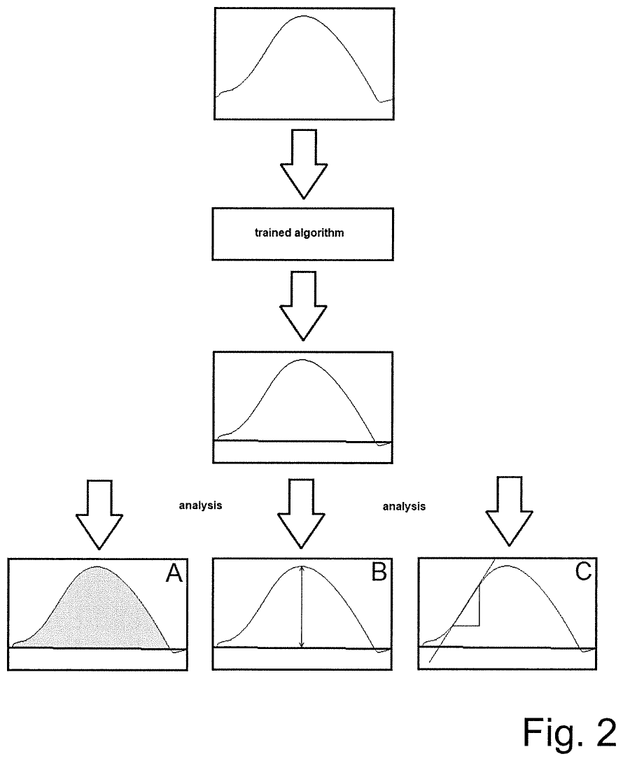

FIG. 3 shows a flowchart of the individual steps of the method for predicting the blood glucose response of an individual to at least one impact factor according to the present invention. According to the method, data pertaining to at least one impact factor are provided and assigned to at least one blood glucose response in a database comprising blood glucose responses of the individual to different impact factors, preferably comprising blood glucose responses of different individuals to different impact factors, in particular comprising blood glucose responses of different individuals classified into the same nutritype to different impact factors, wherein each of the blood glucose responses in the database has been obtained by the method for determining a blood glucose response of an individual to at least one impact factor according to the present invention and consequently has an automatically determined baseline. Subsequently, based on the assignment to at least one blood glucose response in the database a prediction for a blood glucose response to the at least one impact factor in question is provided. FIG. 3 exemplifies an embodiment of the present invention in which the blood glucose responses in the database have been determined by calculating the AUC, in particular the iAUC (grey shaded). Similarly, other methods for the determination of the blood glucose responses in the database, such as determining the maximum increase in the blood glucose level relative to the automatically determined blood glucose baseline or determining the slope of the blood glucose curve are also within the scope of the present invention.

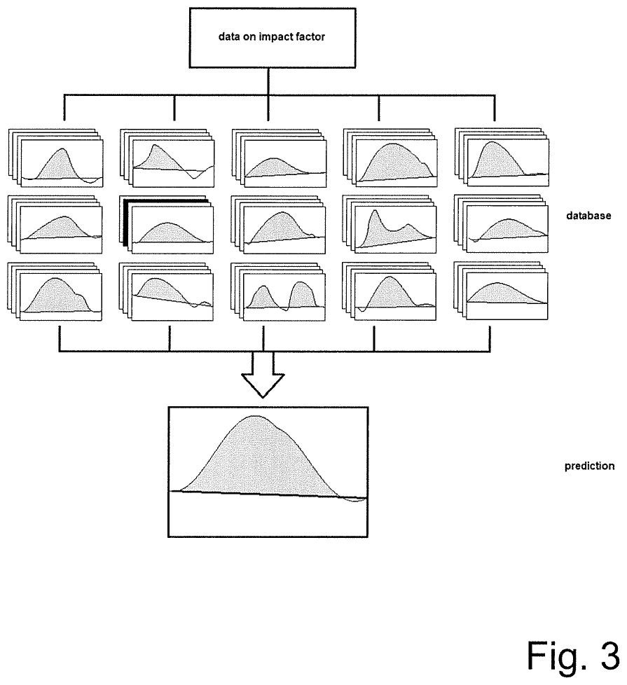

1. A method for determining a baseline in a blood glucose curve, the method comprising:
a) providing training data comprising blood glucose curves of individuals comprising an impact factor-accounting baseline,
b) subjecting the training data provided in step a) to a machine learning procedure to obtain a trained algorithm for automated determination of baselines in blood glucose curves, and
c) determining a baseline in a blood glucose curve by applying the trained algorithm on a blood glucose curve,
wherein the impact factor-accounting baseline in the blood glucose curves of individuals in the training data provided in step a) is a baseline which has been set based on information on the at least one impact factor causative for the deviation of the progression of the blood glucose curve from the blood glucose curve progression which would have been obtained in absence of the at least one impact factor, and
wherein the at least one impact factor is an exogenous or endogenous factor suitable to influence the blood glucose level of an individual.

2. The method of claim 1, wherein the training data provided in step a) contain at least 10, preferably at least 50, preferably at least 100, preferably at least 250, preferably at least 500, preferably at least 750, preferably at least 1000, pairs of blood glucose curves in response to at least one impact factor.

3. The method according to claim 1, wherein the machine learning procedure is a supervised machine learning procedure.

4. The method according to claim 1, wherein the blood glucose curve in step c) is a blood glucose curve of an individual in response to at least one impact factor.

5. The method according to claim 4, further comprising:
d) analysing the blood glucose curve of the individual in response to at least one impact factor having a baseline determined by the trained algorithm, and
e) determining the blood glucose response of the individual to the at least one impact factor.

6. The method according to claim 5, further comprising:
f) assigning the blood glucose response of the individual to the at least one impact factor to a group of blood glucose responses of different individuals to the at least one impact factor in a database using a nutritype classification model, and
g) outputting the nutritype of the individual based on the at least one blood glucose response of the individual to the at least one impact factor.

7. The method according to claim 5, further comprising:
i) providing data pertaining to at least one impact factor,
ii) assigning the data pertaining to at least one impact factor to at least one blood glucose response in a database comprising blood glucose responses of the individual to different impact factors, and
iii) outputting a predicted blood glucose response of the individual to the at least one impact factor.

8. The method according to claim 1, wherein the at least one impact factor is selected from food intake, physical activity, mental activity, stress, health condition medication, sleep or a combination thereof.

9. The method according to claim 5, further comprising
aa) providing data pertaining to at least one impact factor affecting the individual,
bb) assigning the data pertaining to at least one impact factor affecting the individual to at least one blood glucose response in a database comprising blood glucose responses of the individual to different impact factors,
cc) calculating personalized lifestyle recommendations for the individual based on the blood glucose response of the individual to the at least one impact factor affecting the individual in the database of step bb), and
dd) outputting personalized lifestyle recommendations for the individual.

10. The method according to claim 9, wherein the method is a method for determining the composition of a personalized diet, wherein in step cc) the composition of a personalized diet based on the blood glucose response of the individual to the at least one impact factor affecting the individual in the database provided in step bb) is calculated, and wherein in step dd) the composition of a personalized diet is put out.

11. The method according to claim 10, further comprising step ee) of preparing the components of the personalized diet having the composition determined in step cc).

12. A computer program product, directly loadable into the internal memory of a digital computer, comprising software code portions which, when the program is executed by a computer cause the computer to carry out the method according to claim 1.

13. A computer-readable storage medium comprising software code portions, which when executed by a computer cause the computer to carry out the method according to claim 1.

14. A device comprising:
a display unit, displaying a user interface,
an input unit,
a memory unit, and
a processing unit,
wherein the memory unit comprises a computer program product according claim 12.

15. The device according to claim 14, wherein the device is a mobile device, in particular a battery-powered wireless mobile device.

16. A personalized diet obtained by the method of claim 11 for use in the treatment of blood glucose related diseases and/or disorders.

17. The personalized diet for use according to claim 16, wherein the blood glucose related disease and/or disorder is selected from the group consisting of diabetes mellitus type 1 (DMT1), diabetes mellitus type 2 (DMT2), gestational diabetes, hyperglycaemia, metabolic syndrome, cardiovascular diseases, glucose intolerance, polycystic ovary syndrome (PCOS), migraine, non-alcoholic fatty liver disease (NAFLD), cancer, acne, atopic dermatitis, psoriasis, rosacea, atrial fibrillation, dyslipidaemia, HIV, arterial hypertension, pre-diabetes, obesity, brain/cognitive dysfunction, Alzheimer's disease, depression, cartilage damage, Parkinson's disease, rheumatic diseases, chronic inflammation.

18. Use of a personalized diet obtained by the method of claim 11 in induction of pregnancy, regulation of menstrual cycle, weight loss, anti-aging or in treatment and/or prevention of menstrual problems and symptoms of menopause.
